(19) United States
(12) Patent Application Publication
(10) Pub. No.: US 2017/0290493 A1
(43) Pub. Date: Oct. 12, 2017

(54) CARRYING PLATFORM FOR MOVING A DEVICE WITHIN A CONDUIT

(71) Applicant: NANYANG TECHNOLOGICAL UNIVERSITY, Singapore (SG)

(72) Inventors: Assaf COHEN, Singapore (SG); Swee Hin TEOH, Singapore (SG); Huan QI, Singapore (SG)

(73) Assignee: NANYANG TECHNOLOGICAL UNIVERSITY, Singapore (SG)

(21) Appl. No.: 15/509,487

(22) PCT Filed: Sep. 29, 2015
(86) PCT No.: PCT/SG2015/050352

§ 371 Date: Mar. 7, 2017

(30) Foreign Application Priority Data
Sep. 29, 2014 (SG) 10201406164Y

(51) Int. Cl. A61B 1/005; B25J 5/00; A61M 25/00; A61B 1/01; A61M 25/01; B25J 7/00; A61B 1/04

(52) U.S. Cl. CPC A61B 1/0053; B25J 7/00; B25J 5/007; B25J 5/00; A61B 1/0058; A61B 1/0057; A61B 1/04; A61B 1/01; A61M 25/0125; A61M 25/0147; A61M 25/0045; Y10S 901/01; A61M 2205/0266

(57) ABSTRACT

A carrying platform for moving a device within a conduit, the carrying platform comprising: a body configured to be moveable within the conduit; a number of clampers provided on the body and configured to releasably engage the conduit for immobilizing the carrying platform relative to the conduit; and a device engagement mechanism provided on the body and configured to releasably engage the device.

### FIELD OF THE INVENTION

This invention relates to a carrying platform for moving a device such as a catheter within a conduit such as a blood vessel or other biological lumen.

### BACKGROUND OF THE INVENTION

Currently, catheters lack the ability to move deep inside narrow vessels, and at curvatures, risk damaging or even puncturing the vessel. Thus, it is desirable to have a standalone device which can be used together with existing catheters or scopes to assist doctors in navigating narrow vessels.

### SUMMARY OF INVENTION

The carrying platform comprises a body having a longitudinal axis and a radial outer and inner surface. The body has a through hole along the longitudinal axis configured to receive a device with an elongate body therethrough, such as a catheter. The carrying platform has spaced apart external clampers on the radial outer surface, and an actuating mechanism which forms part of the hollow tubular body that allows the body to selectably extend or contract. Grippers that are spaced apart on the body are configured to be selectively activated to grip the device.

According to a first aspect, there is provided a carrying platform for moving a device within a conduit, the carrying platform comprising: a body configured to be moveable within the conduit; a number of clampers provided on the body and configured to releasably engage the conduit for immobilizing the carrying platform relative to the conduit; and a device engagement mechanism provided on the body and configured to releasably engage the device.

The device engagement mechanism may comprise at least a first set of wheels configured to effect relative movement between the carrying platform and the device and to allow the carrying platform to move along a length of the device within the conduit.

The body may comprise an actuating mechanism configured to selectably extend and contract the body; the number of clampers may comprise a front clamper and a rear clamper provided spaced apart on the body, the front clamper and the rear clamper each configured to independently engage the conduit such that a front portion of the body is immobilised relative to the conduit when the front clamper engages the conduit and a rear portion of the body is immobilised relative to the conduit when the rear clamper engages the conduit; and the device engagement mechanism may comprise a front gripper and a rear gripper provided spaced apart on the body, the front gripper and the rear gripper each configured to independently grip the device.

The carrying platform may be configured such that engaging the rear clamper with the conduit and extending the body when the front clamper is disengaged with the conduit results in forward movement of the front portion in the conduit, wherein engaging the rear clamper with the conduit and contracting the body when the front clamper is disengaged with the conduit results in backward movement of the front portion in the conduit, wherein engaging the front clamper with the conduit and extending the body when the rear clamper is disengaged with the conduit results in backward movement of the rear portion in the conduit, and wherein engaging the front clamper with the conduit and contracting the body when the rear clamper is disengaged with the conduit results in forward movement of the rear portion in the conduit.

When the rear gripper is not gripping the device, movement of the front gripper in the conduit when gripping the device may correspondingly move the elongate device in the conduit; and when the front gripper is not gripping the device, movement of the rear gripper in the conduit when gripping the device may correspondingly move the device in the conduit.

The front gripper and the rear gripper may each comprise one of: a sleeve made of a shape memory material, a sleeve actuated by wire to grip the elongate device, and a balloon configured to grip the device when inflated.

The body may have an inflatable wall comprising an external elastic layer and an internal elastic layer defining a space therebetween, wherein the external elastic layer is stiffed with fibres and the internal elastic layer is stiffened by a spring, and wherein the internal elastic layer defines a hollow passage through the body.

The front clamper may be provided with pressure supply lines that pass through the hollow passage, the pressure supply lines configured to cause bending of the body to occur when the inflatable wall is inflated and one of the pressure supply lines is pulled or held in place.

The carrying platform may further comprise an imaging modality that passes through the hollow passage.

The body may be hollow and may comprise a bellow.

The body may comprise a cylinder actuator having a piston-in-cylinder configuration, wherein the front clamper is provided on the piston and the rear clamper is provided on the cylinder.

The front clamper and the rear clamper may each comprise a balloon configured to engage the conduit when inflated.

Alternatively, the front clamper and the rear clamper may each comprise arms that engage the conduit when at rest, the arms being actuated by a wire to disengage with the conduit when the wire is pulled.

Alternatively, the front clamper and the rear clamper may each comprise arms that are disengaged with the conduit when at rest, the arms being actuated by a wire to engage with the conduit when the wire is pulled.

The carrying platform may further comprise a spring provided in the body wherein pulling a further wire compresses the spring to contract the body.

Gecko adhesive may be provided on the number of clampers at where the number of clampers are configured to engage the conduit.

The carrying platform may be configured to be connected to a further carrying platform of any preceding claim.

The carrying platform may be covered with an elastic layer configured to protect the carrying platform from substances found within the conduit.

### BRIEF DESCRIPTION OF FIGURES

In order that the invention may be fully understood and readily put into practical effect there shall now be described by way of non-limitative example only exemplary embodiments of the present invention, the description being with reference to the accompanying illustrative drawings.

FIG. 1(a) is a schematic sectional view of an exemplary embodiment of the carrying platform of the present invention carrying a device.

FIG. 1(b) is a schematic sectional view of an exemplary embodiment of the carrying platform of the present invention carrying a device.

FIG. 2 is a schematic sectional view of the carrying platform of FIG. 1 fully gripping the device in a lumen and movable within a lumen by moving the device.

FIG. 3 is a schematic sectional view of the carrying platform of FIG. 1 fully engaging a lumen without gripping the device.

FIGS. 4(a) to 4(d) are schematic sectional views of the carrying platform of FIG. 1 at different stages of movement over the device within a lumen.

FIGS. 5(a) to 5(d) are schematic sectional views of the carrying platform of FIG. 1 at different stages of movement in moving the device within the lumen.

FIGS. 6(a) to 6(d) are schematic sectional views of the carrying platform of FIG. 1 at different stages of movement in moving the device within the lumen.

FIGS. 7(a) to 7(d) are schematic sectional views of the carrying platform of FIG. 1 at different stages of movement in moving the device within the lumen.

FIGS. 8(a) to (d) are schematic perspective views of stages of manufacture of the carrying platform.

FIGS. 9(a) to (f) are schematic perspective views of stages of mounting the carrying platform to a catheter.

FIG. 10 is a schematic perspective view of the carrying platform having flexible hinges.

FIGS. 11(a) and (b) are schematic perspective views of two alternative on-piston load bearing configurations: rear load bearing and front load bearing.

FIG. 12 is a schematic perspective cut-away view of another exemplary embodiment of the carrying platform with fibre and spring stiffening extensor.

FIG. 13 is a schematic perspective cut-away view of a further exemplary embodiment of the carrying platform with a front clamper inlet passing through the body which is comprised of bellows.

FIG. 14 is schematic cross-sectional views of the carrying platform having additional clampers configured for manipulating its movement.

FIG. 15 is a photograph of the carrying platform with confined luminescent material sealed-off between its clampers.

FIGS. 16(a) to (c) are schematic perspective views of the carrying platform actuated by wires.

FIGS. 17(a) and (b) are schematic sectional views of an alternative carrying platform actuated by cables.

FIG. 18 is a perspective view of an exemplary wire actuated carrying platform.

FIG. 19 is a perspective view of a further alternative wire actuated carrying platform with clampers comprising bendable rods.

FIG. 20 is a perspective view of the carrying platform of FIG. 19 covered with an elastic layer and deployed in a blood vessel.

FIG. 21 is a perspective view of three variations of exemplary clampers of the carrying platform.

FIG. 22 is a perspective view of a further exemplary carrying platform of the present invention.

FIG. 23 is a graph of load vs pressure for gecko-patterned and non-patterned silicone

### DETAILED DESCRIPTION

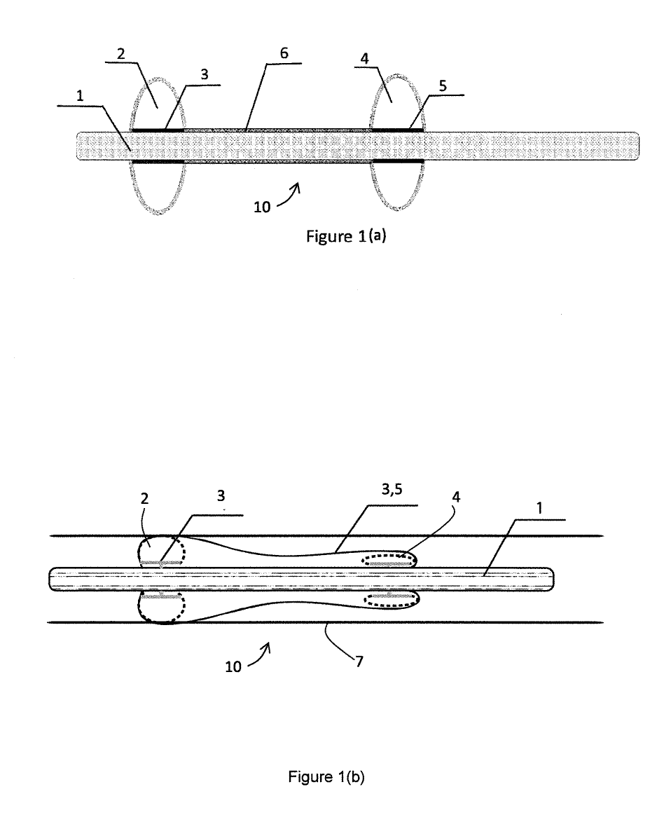

Exemplary embodiments of the carrying platform 10 for moving a device 1 within a conduit will be described below with reference to FIGS. 1 to 23, in which the same reference numerals are used to denote the same or similar parts.

In general, the carrying platform 10 comprises a body 6 configured to be moveable within the conduit 7, and a number of clampers 2, 4 provided on the body 6 and configured to releasably engage the conduit 7 for immobilizing the carrying platform 10 relative to the conduit 7. The carrying platform 10 also comprises a device engagement mechanism provided on the body 6 and configured to releasably engage a device 1 within the conduit to effect relative movement between the carrying platform 10 and the device 1.

In a preferred embodiment, the carrying platform 10 is configured to be secured to the device 1 to ensure that it does not ever come off the device 1 particularly when the carrying platform 10 is inside a human body.

In one embodiment, as shown in FIG. 1(a), the carrying platform 10 comprises a tubular body 6 having a longitudinal axis, a radial outer surface and a radial inner surface. The body 6 has a through hole along the longitudinal axis configured to receive a device 1 with an elongate body therethrough, such as a catheter 1. The carrying platform 10 has spaced apart external front and rear clamping devices or clampers 2, 4 respectively provided on the radial outer surface of the body 6. An actuating mechanism (as will be described in greater detail below) forms part of the hollow tubular body 6 that allows the body 6 to selectably extend or contract according to actuation by a user. The carrying platform 10 also has internal front and rear grippers 3, 5 respectively provided spaced apart in the inner radial surface of the body that serve as the device engagement mechanism 3, 5. The front and rear grippers 3, 5 are configured to independently grip the device 1 as actuated by a user.

The front and rear clampers 2, 4 may each take the form of a balloon such that inflation of the balloon results in the clamper 2 or 4 engaging the conduit 7 in which the carrying platform 10 is placed.

The front and rear grippers 3, 5 are configured to grip the device 1 and may each take the form of a sleeve, a bulge, a spring, a membrane, an electromagnetic actuator, shape memory material, bimetal actuators, hydraulic or pneumatic actuators, electroactive polymers a noose, wire, strings, rotational wheels, or any form or configuration that allows controllable attachment and detachment of the carrying platform 10 with the device 1. This may or may not be selective to a specific location of the carrying platform 10 or the device 1. The sleeve may be made of a shape memory material, or wires may be provided to tighten the sleeve to actuate gripping. Alternatively, the front and rear grippers 3, 5 may each comprise a balloon such that inflation of the balloon results in the gripper 3 or 5 gripping the device 1. Activation or deactivation of the clampers 2 and 4 may be directly or indirectly coupled to the activation or deactivation of the grippers 5 and 3 respectively.

The front and rear clampers 2, 4 are configured such that a front portion of the body where the front clamper 2 is provided is immobilised relative to the conduit 7 when the front clamper 2 engages the conduit 7, and a rear portion of the body where the rear clamper 4 is provided is immobilised relative to the conduit 7 when the rear clamper 4 engages the conduit 7. Thus, the carrying platform may be said to comprise three sections or portions—a front portion comprising the front clamper 2 and front gripper 3, a rear portion comprising the rear clamper 4 and rear gripper 5, and the body 6 being a third portion that can be extended and contracted.

In one embodiment, the front clamper 2 and the rear clamper 4 each includes at least one gripper, the front gripper 3 and the rear gripper 5 respectively. The two clampers 2, 4, and two grippers 3, 5 are connected with the body 6.

When the carrying platform 10 is in use, the length of the extension part or body 6 can be adjusted in a way to cause the carrying platform 10 to extend or contract, resulting in forward or backward motion, with or without the movement of the device 1 itself, as will be described below. Note that this locomotion technique, traditionally termed “inchworm locomotion,” is exemplary, and can be replaced by any other locomotion technique such as earthworm, snake, jet, amoeboid, wheels based, and any other locomotion technique that is suitable for carrying or stabilizing the device 1 inside the conduit 7. For example, as shown in FIG. 1(b), the carrying platform 10 is engaging a device comprising a catheter 1 via a set of rotational wheels 3 that serve as the device engaging mechanism when the catheter 1 is inserted through a central longitudinal through hole of the carrying platform 10. The set of motorised wheels 3 can be made of silicone which is soft and can provide gripping force when it is engaged. The through hole is made slightly bigger than the diameter of the carrying platform so that it can be easily slotted in. The motorised wheels 3 can be activated to engage and grip onto the catheter 1. By having at least one set of motorised wheels 3 engaging the catheter 1 (preferably two sets 3, 5 of motorized wheels as shown in FIG. 1(b)), the carrying platform 10 can be controlled to move along the length of the catheter 1 within the conduit 7. In alternative embodiments, instead of motorised wheels, other forms of manipulators such as micro-robotics legs may be provided to enable similar movement of the carrying platform 10 along the length of the catheter 1 within the conduit 7. In this configuration, extension and contraction of the body 6 will not be required to effect movement of the carrying platform 10.

Alternatively, forward motion of the carrying platform 10 can be achieved by inflating the first balloon or rear clamper 4 to engage or establish a grip on the wall or internal surface of a lumen 7 in which the carrying platform 10 is placed before extending the actuating mechanism to advance the body 6. After extending the body 6, the first balloon 4 is deflated while the second balloon or front clamper 2 is inflated to engage or establish a grip on the far end or internal surface of the lumen 7. The actuating mechanism then retracts and contracts the body 6, thereby moving the first balloon 4 forward. Repeating the above sequence of events moves the carrying platform 10 forward in the lumen. Appropriate use of the internal front and rear grippers 3, 5 that can be activated to grip the device 1 thus allow the carrying platform 10 to move the device 1 along the lumen during the forward motion. Various possibilities of motion can be achieved with the independent activation of the internal grippers 3, 5.

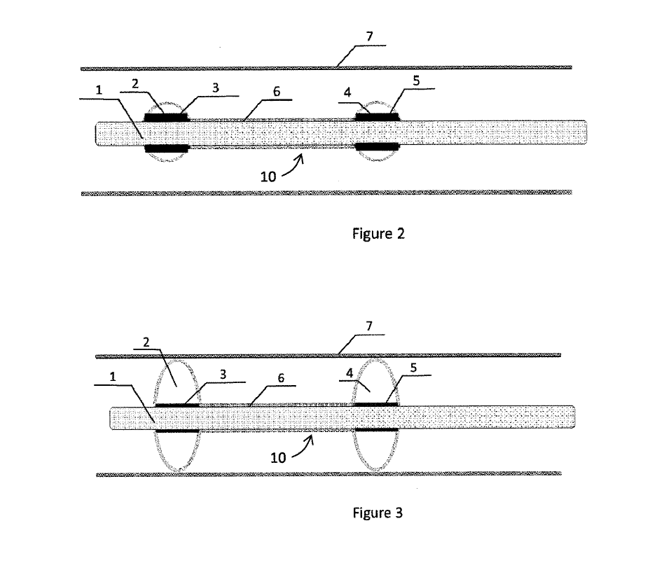

FIG. 2 and FIG. 3 illustrate a sectional view of the carrying platform 10 in a rest status according to the present invention, in which the device 1 is operated or moved within the lumen without the active assistance of the carrying platform 10.

In a first instance as shown in FIG. 2, both the front gripper 3 and rear gripper 5 are activated to grip the catheter 1, while both the front clamper 2 and rear clamper 4 are deactivated and disengaged with the internal surface of the lumen 7. Alternatively, only either the front gripper 3 or the rear gripper 5 is activated to grip the device 1 at one location. In either case, the carrying platform 10 is attached to the device 1 while being disengaged with the conduit 7, and the carrying platform 10 will move together with the catheter 1 similarly to conventional standard-of-care catheters, by active pulling or pushing of the catheter 1 by a user such as a surgeon from the outside of the patient.

In a second instance as shown in FIG. 3, both the front gripper 3 and rear gripper 5 are deactivated and not gripping the catheter 1 and both the front clamper 2 and rear clamper 5 are activated to engage the internal surface of the lumen 7. Thus, the carrying platform 10 will temporarily attach to the lumen wall or conduit 7. In this case, the catheter or device 1 can function or be moved while the carrying platform 10 works as a stabilizer to hold the catheter 1 at a certain position or serve another purpose such as reducing the friction experienced by the endothelium of a vessel. For example, the carrying platform 10 can be used as a guide for the catheter 1 when moving inside narrow vessels, and at curvatures, reducing the risk of the catheter 1 damaging or even puncturing the vessel that may be caused due to high friction due to direct contact of the catheter with the vessel that will exist without the carrying platform 10. Moreover, certain application such as localized drug delivery can be realized between the two clampers 2, 4. For example, during the catheterization process, if a plaque is detected during the process, the carrying platform 10 may be deployed to release a drug while the device 1 continues to advance until another plaque is encountered and then the next in line carrying platform 10 is deployed and the process can be repeated. It is envisaged that additional clampers (not shown) may be provided on the carrying platform 10 for larger treatment areas.

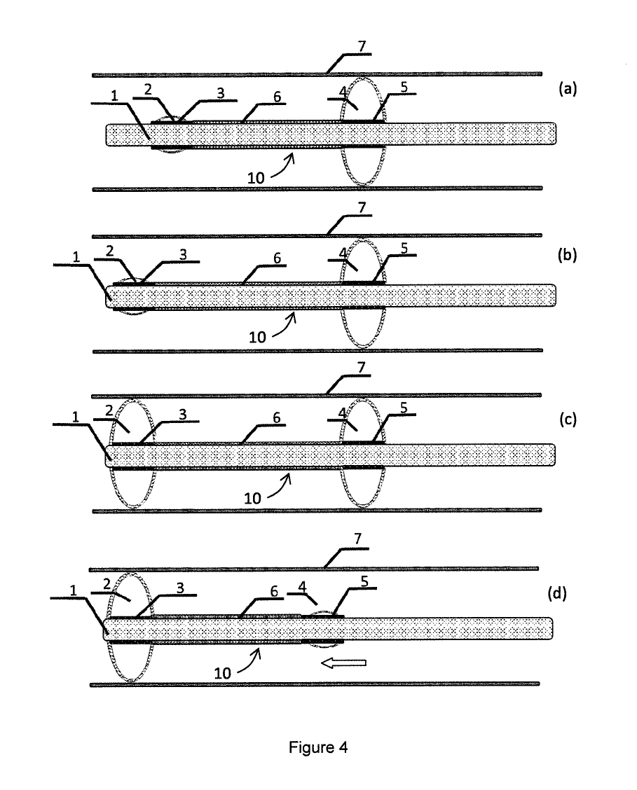

FIG. 4 shows the carrying platform 10 operating autonomously, in moving forward or backward to a certain position without moving the catheter 1 or other elongate device 1. In this situation, both the front gripper 3 and rear gripper 5 are deactivated and not gripping the catheter 1, thus, the carrying platform 10 is functionally detached from the catheter 1. This means that the catheter can move forward and backward without the assistance of the catheter carrying platform. In operation, first, as shown in FIG. 4(a), the front clamper 2 is deactivated and disengaged from the conduit 7 and the rear clamper 4 is activated to engage the conduit 7, thereby attaching the rear portion of the carrying platform 10 to the conduit 7. The rear portion is thus immobilized relative to the lumen 7. Subsequently, as shown in FIG. 4(b), the extension part or body 6 is extended, moving the front part or portion of the carrying platform 10 forward relative to both the conduit 7 and the catheter 1. Afterward, as shown in FIG. 4(c), the front clamper 2 is activated to engage and attach to the lumen wall or conduit 7. The front portion is thus immobilized relative to the conduit of the lumen. This is followed by the rear clamper 4 being deactivated and disengaged from the conduit 7 as shown in FIG. 4(d). At this point, the extension part or body 6 being contracted results in movement of the rear part or portion of the carrying platform 10 forward in the direction shown by the arrow. By repeating the above steps, the carrying platform 10 will be able to move forward (or backward) along the catheter 1 and relative to the conduit 7 without moving the catheter 1 itself. In this embodiment, the carrying platform 10 operates by applying pressure to the wall 7. This can be used for several applications such as mapping the stiffness, diameter, and/or shape of the arteries by sensors provided within the clampers 2, 4; releasing calcified plagued or blocked vessels by application of pressure; at stage 4(c) reactive solution can be infused within the sealed-off gap formed by the clampers 2, 4 to treat/rejuvenate the vessel at each step.

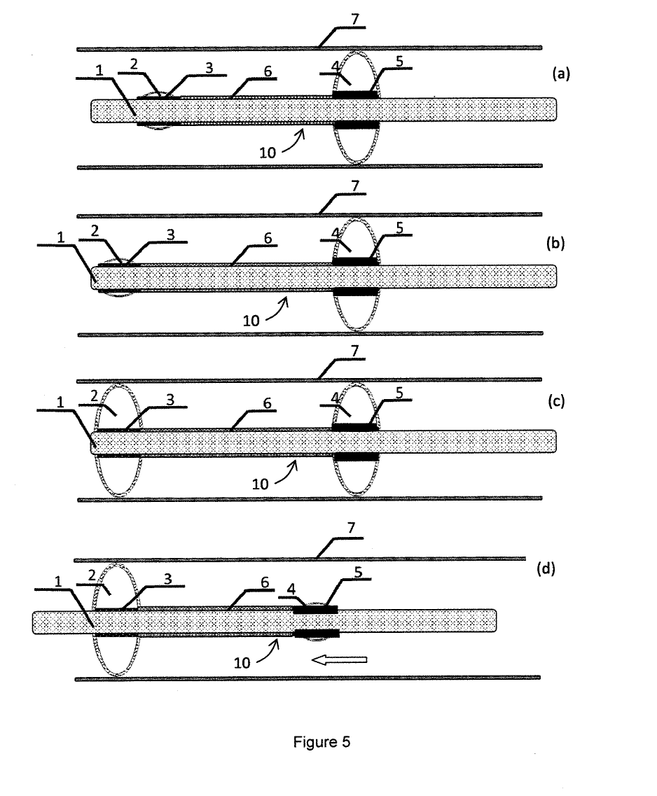

FIG. 5 shows the carrying platform 10 operating together with the catheter 1 to move the catheter 1 forward in the conduit 7 in the direction shown by the arrow. Throughout the operation, the front gripper 3 is kept deactivated and not gripping the catheter 1. Thus, the front clamper 2 and front section of the carrying platform 10 is able to move freely relative to the catheter 1. The rear gripper 5 is kept activated to grip the catheter 1, fastening the rear section of the carrying platform 10 to the catheter 1. In use, first, as shown in FIG. 5(a), the front clamper 2 is deactivated and disengaged from the conduit 7 and the rear clamper 5 is activated, thereby engaging the conduit 7 and attaching the rear portion of the carrying platform 10 to the lumen wall 7. The rear portion is thus immobilized relative to the conduit 7 of the lumen. Subsequently, as shown in FIG. 5(b), the extension part or body 6 is extended, moving the front part or portion of the carrying platform 10 forward. Afterward, as shown in FIG. 5(c), the front clamper 2 is activated to engage the conduit 7 and attach to the lumen wall 7. The front portion is thus immobilized relative to the space of the lumen. Finally, as shown in FIG. 5(d), the rear clamper 5 is deactivated to disengage with the conduit 7 while the extension part or body 6 is contracted, moving the rear part or portion of the carrying platform 10 together with the catheter 1 forward. Repeating the same process, the carrying platform will move forward (and similarly, backward), carrying the catheter 1 along the lumen. This can be used to propel a catheter within the vessel with less frictional force exerted onto the endothelium. Moreover, this allows navigation with significantly less frictional force, especially at curvatures. If this locomotion add-on carrying platform is added to an endoscope or a catheter and its locomotion power is added to work in tandem with the pushing force exerted by the surgeon, motion of these devices will become faster, safer and more efficient. Moreover, the most important novelty of the carrying platform is that this is an add-on device that is not intended to replace current standard catheters. The embodiments of the carrying platform 10 described herein are added to existing products in situ and on a when-needed basis, thus allowing use of this invention with standard market products and procedures. This allows the users to keep all the advantages of current products while adding new features and advantages that are made possible by the invention described herein.

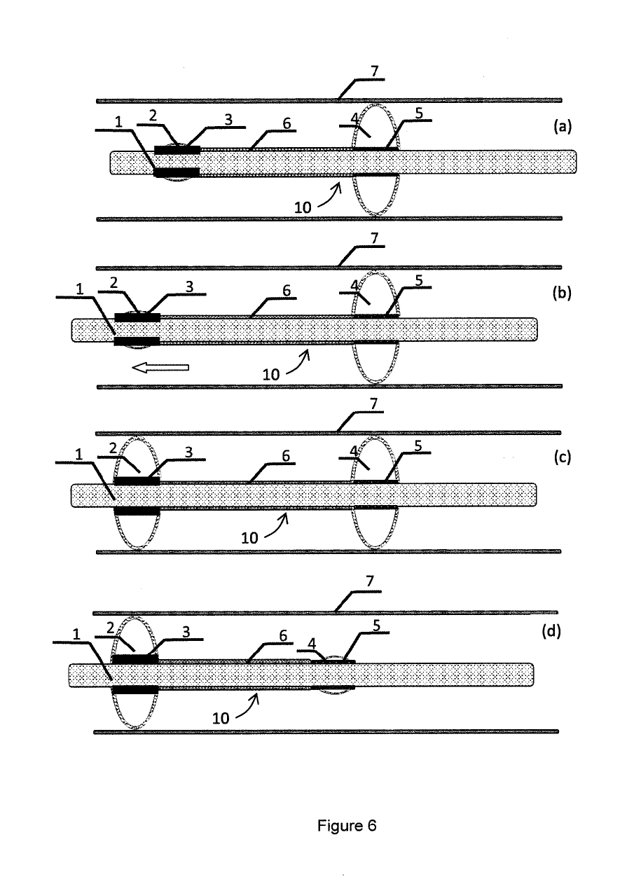

FIG. 6 shows the carrying platform 10 operating together with the catheter 1 to move the catheter 1 forward in the conduit 7 in the direction shown by the arrow. Throughout the operation, the front gripper 3 is kept activated to grip the catheter 1, fastening the front section of the carrying platform 10 to the catheter 1. Thus, the front clamper 2 and front section of the carrying platform 10 moves together with the catheter 1. The rear gripper 5 is kept deactivated and not gripping the catheter 1. In use, first, as shown in FIG. 6(a), the front clamper 2 is deactivated and disengaged with the conduit 7 and the rear clamper 4 is activated to engage the conduit 7, attaching the rear portion of the carrying platform 10 to the lumen wall 7. The rear portion is thus immobilized relative to the conduit 7 of the lumen. Subsequently, as shown in FIG. 6(b), extension part or body 6 is extended, moving the front part or portion of the carrying platform 10 forward together with the catheter 1. Afterward, as shown in FIG. 6(c), the front clamper 2 is activated to engage the conduit 7, attaching to lumen wall 7. The front portion is thus immobilized relative to the conduit 7 of the lumen. Finally, as shown in FIG. 6(d), the rear clamper 4 is deactivated to disengage with the conduit 7 while the extension part 6 contracts, moving the rear part or portion of the carrying platform 10 forward without movement of the catheter 1. Repeating the same process, the catheter carrying platform 10 will move forward (and similarly, backward) together with the catheter 1 along the lumen. In contrast with FIG. 5, which uses rear load bearing, in FIG. 6, the carrying platform 10 utilizes front load bearing for propelling the device 1. This allows better motion performance in some cases, for example when the device 1 is unable to move when rear load bearing is exerted. However, the main reason for choosing between rear and front load bearing (or middle bearing (not mention for other locomotion techniques or for this inchworm) is dependent on the type of actuator or locomotion technique used. If the actuator is stronger when contracting than it is when expanding, rear load bearing will be used. If the actuator is stronger in extension that in contraction, front load bearing will make more sense. The choice of load bearing position can also alter if the device 1 is within a curvature, both to avoid device 1 hitting the walls or to allow easier sliding of device 1 within the carrying platform 10.

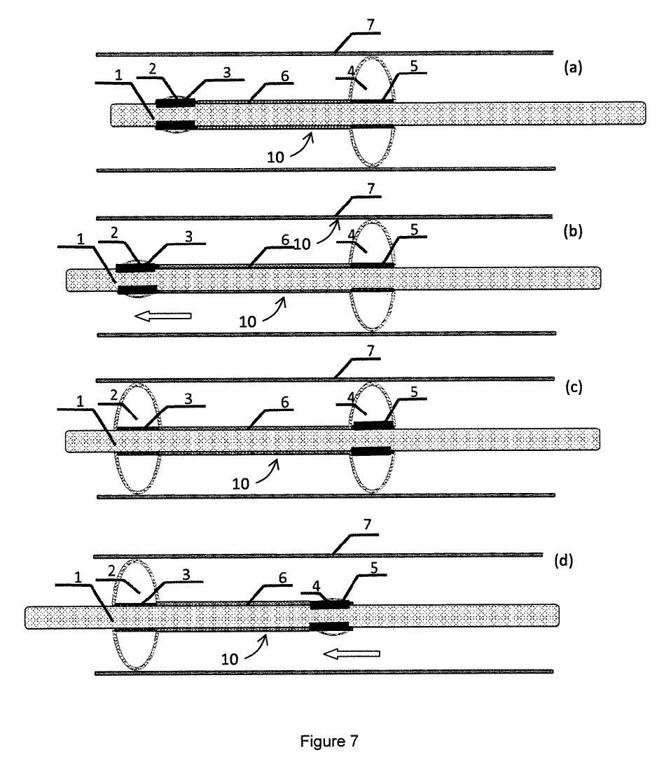

FIG. 7 shows the carrying platform 10 operating to move the catheter 1 forward in the conduit 7 in the direction shown by the arrow. Initially, as shown in FIG. 7(a), the front gripper 3 is activated to grip the catheter 1 while the clamper 2 is deactivated and disengaged with the conduit 7, thus the front part or portion of the carrying platform 10 can move forward together with the catheter 1. The rear clamper 4 is activated to engage the conduit 7, attaching the catheter carrying platform to the lumen wall 7. The rear portion is thus immobilized relative to the conduit 7 of the lumen. Meanwhile, the rear gripper 5 is deactivated and not gripping the catheter 1. Thus, the catheter 1 can move together with the front clamper 2. Subsequently, as shown in FIG. 7(b), extension part or the body 6 is extended, thereby moving the front part or portion of the carrying platform 10 together with the catheter 1. Afterward, as shown in FIG. 7(c), the front clamper 2 activated to engage the conduit 7 and attach to lumen wall 7. The front portion is thus immobilized relative to the conduit 7 of the lumen, while the front gripper 3 is deactivated and the rear gripper 5 activated to grip the catheter 1. Finally, as shown in FIG. 7(d), the rear clamper 4 is deactivated to disengage with the conduit 7 while the extension part or body 6 is contracted, thereby moving the rear part or portion of the carrying platform 10 together with the catheter 1. Repeating the same process, the carrying platform 10 is able to move forward (as well as backward) carrying the catheter 1 along the lumen at double the speed of the configurations shown in FIGS. 5 and 6, meanwhile allowing changing its position in relation to the catheter 1 at the same time.

While the motion cycle described above with reference to FIGS. 4-7 have been presented as four separate defined steps (a) to (d) in each of the FIGS. 4-7, it is important to note that these steps can be varied in order and combination. In fact, during actual operation, two steps may be actually performed at once as the steps can be performed in parallel.

In addition, as shown in FIG. 3, both or one clamper 2 or 4 can be actuated to engage the conduit 7. In this application, device 1 is stabilized within the vessel. Thus, if the grippers 3, 5 are in the “off” positions, the device 1 can be moved by application of external force, sliding along the internals of the carrying platform 10. This allows reduced friction between device 1 and the lumen walls, especially useful in torturous vessels, curvatures and narrowing. Moreover, in this application, if the grippers 3, 5 and clampers 2, 4 and extensor/contractor midsection 6 are operated in the correct sequence, the device 1 can be propelled within the vessel without forward motion of carrying platform 10.

Another important application arising from this design can be seen in FIG. 2. Here, both clampers 2, 4 are in the “off” position. Thus, by using the internal grippers 3, 5 alone, the carrying platform 10 can “walk” on top of the device 1 without touching the wall 7. Thus, the carrying platform 10 can change its location relative to the device 1, navigating to areas in which it is needed. In applications such as brain surgery for example, or in applications where the carrying platform 10 is too large and unable to be inserted into the lumen, the carrying platform 10 can still be used to assist in propelling device 1 from a location other than the tip of the device 1.

To allow for a higher navigation capability of the carrying platform 10, and in particular to increase pulling force on the elongate device 1, several carrying platforms 10 can be used simultaneously, for example, either by being chained together or attached or provided at intervals along the device 1. The connected carrying platforms 10 can either advance together with the device 1 or be detached from it at some point while continuing to propel the device 1 from its midsection along the desired path, or simply be deployed locally to reduce the friction between device 1 and walls 7.

When the space to be navigated comprises a larger divergence of distances between the wall or conduit and the carrying platform 10, i.e., when cross-sectional area of the conduit varies, for example when moving in both smaller and larger arteries), it is envisaged that the elongate device 1 to be moved may be equipped with carrying platforms 10 of different sizes to be utilized at different locations along the navigated path, according to the required diameter or distance of the conduit, to facilitate movement of the elongate device 1 in the conduit of varying internal cross-sectional area. Likewise, the carrying platform 10 may be equipped with several kinds of clampers for each diameter or gap range and for other functions. For example, clampers that are more suitable for force opening clogged vessels, clampers that are more suitable for sealing-off a portion of vessel for drug delivery, clampers that allow continuation of blood flow, clampers that include a photocatalytic function, clampers that have an active agent, such as drug emitted or embedded in them, clampers that can better sense properties of the environment or the wall, and so on. Likewise, a single clamper may be fitted to exhibit any one of these traits separately or at the same time.

Another possibility is to include more than one extensor or body 6, or more than two clampers 2, 4 in the carrying platform 10, in order to provide greater stability, higher force etc. Providing several carrying platforms at locations along the catheter line is especially useful as a larger diameter carrying platform is cheaper to produce which means that the carrying platform can be positioned along the line where the vessel diameter is larger, whereupon it will push the catheter from a point along the line where the carrying platform is located through the blockage.

To overcome blockages in a vessel, the extensor or body 6 and front clamper 2 can be repeatedly actuated, to aid navigation of the carrying platform 10 in the conduit. This ramming of clots, together with active chemicals or drugs that can be secreted, can be used to breakdown blockages. In addition, vibrations may be produced by the carrying platform 10 in order to overcome vessel blockage, while expandable (stent-like) rings or other similar structures can be deployed from the carrying platform 10 when needed. Said rings or structures can be biodegradable. It is also envisaged that secretion of dissolving or other active agents can be performed by the carrying platform 10 to clear blockage in the conduit.

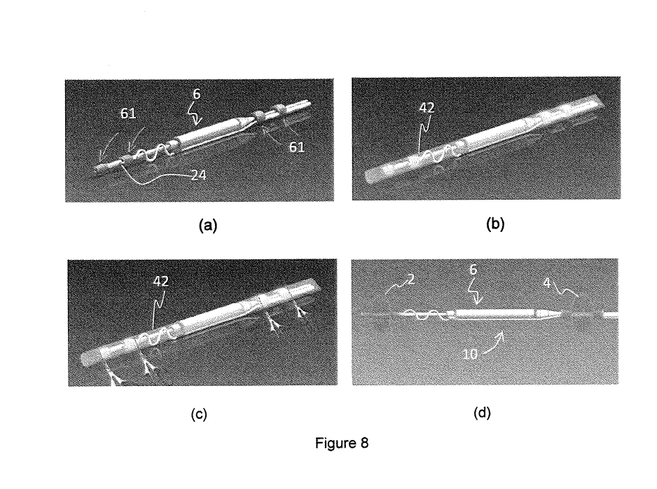

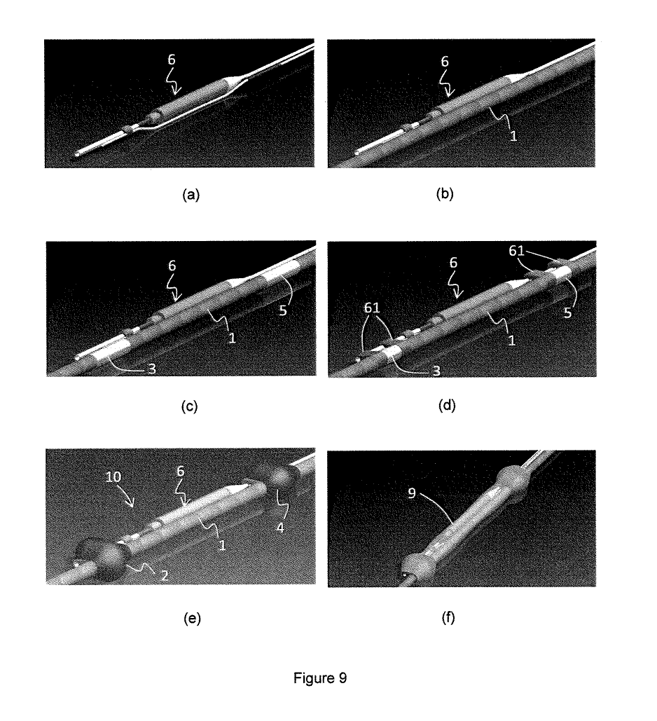

One possible manufacturing process of the carrying platform 10 is illustrated in FIGS. 8(a) to (d). The body 6 is prepared by first providing an extensor or actuator, such as a cylinder actuator having a piston-in-cylinder configuration, and then using pairs of epoxy castings 61 (or any other suitable material) to attach inlets and outlets 24 of the balloon clampers 2, 4 to the main body 6, as shown in FIG. 8(a). Several such main bodies 6 are afterwards inserted into a tube 42 which is made of stretchable material, with or without certain pre-stretch of said tube 42, to form several carrying platforms 10. FIG. 8(b) shows one main body 6 inserted into a tube 42. Pre-stretch can be used to increase “stiffness” of the balloons 2, 4 formed therewith, resulting in faster deflation. Likewise, controlled leak holes can be introduced near the inlets 24 or on the balloons 2, 4 to assist in faster deflation as the saline solution used for inflation can leak via these holes to increase speed of deflation. Moreover, if motion steps are initialized before full deflation/inflation, the motion attempt will also contribute to faster deflation as additional force will be exerted on the balloons 2, 4. The pairs of epoxy castings 61 are glued to the tube using injection of a low viscosity epoxy adhesive or heat or UV activated adhesive. Excess of the tube may be cut away if desired, as shown in FIG. 8(c), revealing the balloons 2, 4 each formed between each pair of epoxy castings 61 as shown when inflated in FIG. 8(d). Alternatively, by not cutting away the excess tube, return force is increased.

One possibility of implementation of mounting the carrying platform 10 to a catheter 1 or other elongate device 1 is illustrated in FIGS. 9(a) to (f). The only difference with the process described above with reference to FIGS. 8(a) to (d) is that the epoxy castings 61 are also engulfing two sleeves 3, 5 onto which the catheter 1 can be mounted, as shown in FIG. 9(d). These sleeves 3, 5 can serve as the front and rear grippers 3, 5. This can be achieved for example by using shape memory material, bimetal strips, pulling wires to tighten the sleeves 3, 5, using membranes or balloons, electromagnetic or any other actuator (not shown) may be added to serve as the front and rear grippers 3, 5. An elastic layer 9, which can be biocompatible and incorporate features such as gecko or treefrog adhesive structures, drug eluting materials, anticoagulant coating, omniphobic or superhydrophobic coating etc. may further be added to separate the carrying platform 10 from the conduit 7. The gap between the catheter 1 and the clampers 2, 4 may be functionalized with omniphobic or superhydrophobic or other coating to prevent blood from penetrating into the interior of the carrying platform 10.

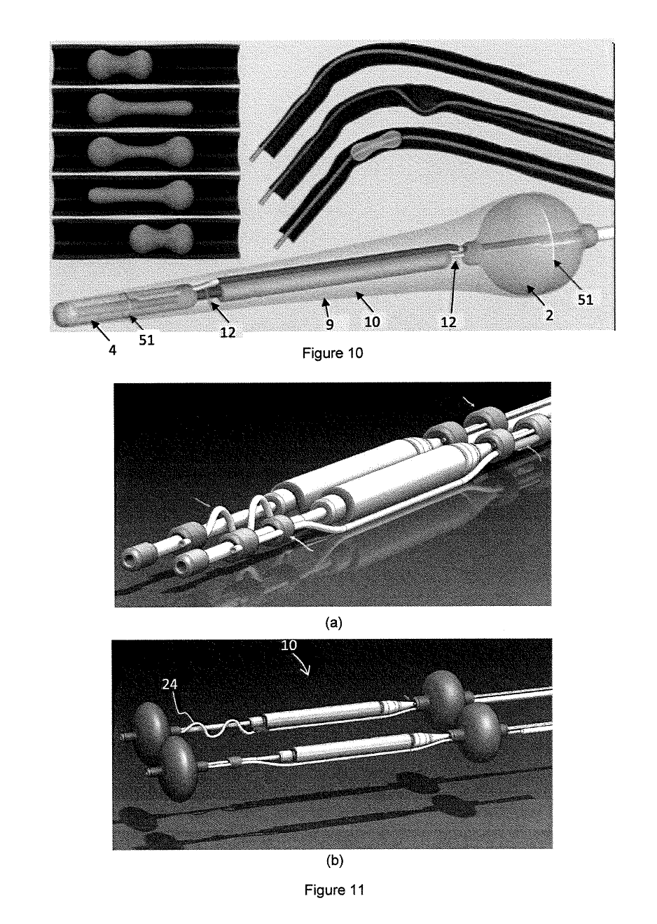

To improve navigation capability of the carrying platform 10, flexible hinges 12 that allow bending of the carrying platform 10 may be placed at edges of the extensor or body 6, whether or not the extensor or body 6 is itself flexible, as shown in FIG. 10. This improves mobility of the carrying platform 10, especially in curved vessels, in order to allow the carrying platform 10 to bend and conform to a certain degree of curvature of the conduit. Also, a type of special extensor or body 6 that uses spring embedded in elastic material can also be used for the body 6. This can be further developed to create the hollow extensor or body 6, but can also be used to fabricate many other types of extensors or body 6 for various embodiments of the carrying platform 10. In addition, as shown in FIG. 10, strain sensors 51 may be provided on the inflatable clampers 2, 4.

Encapsulating, or coating the carrying platform with a thin coating, layer or sheet of elastic material 9 as shown in FIG. 10 can improve the performance, lifetime, and reduce production costs of the carrying platform by insulating the carrying platform from the environment, enabling use of cheaper materials for fabricating the carrying platform and easier sterilization etc. Furthermore, this coating in itself can be coated/embedded with an active agent, such as heparin or other anticoagulants, while the carrying platform is being used as a catheter. The sheet may also contain outlets for supply channels that may be supplying, for example, active ingredients. The piston uses hydraulic pressure to extend but the force from a return spring to retract. The force of the spring is 0.5N and the force of the pressure is 1.9N. As the return force of the piston of the body 6 is significantly weaker than its pressure-dependent advancing force, it is more desirable to link the load (i.e. the catheter 1) directly to the piston rod rather than to its tail. To achieve that, the front balloon inlet 24 is inserted via a channel that passes inside the rear balloon, as shown in FIG. 11.

Another possibility when using a piston extensor for the body 6 is to orientate the piston extensor with the cylinder portion forward and the piston rod rearward such that the rear clamper 4 is provided on the piston rod while the front clamper is provided on the cylinder, so as to allow for a greater pushing force.

To allow bending of the carrying platform 10, it is possible to incorporate a nonconcentric balloon, that is, a balloon that is thicker or stiffer on one side, as one or both of the front and rear clampers 2, 4, in order to have a bending, or a bending-extension motion. It is also possible to embedded stiffness adjustable materials that can change their stiffness in response to electrical stimuli of other stimuli alternatives. It is also possible to embed fibers or springs inside or outside the balloon walls of the front and/or rear clampers 2, 4 in order to induce an axial or bending motion. Using springs will have the added benefit of increasing mechanical stability and return force.

The carrying platform 10 described in the above embodiments describe use of balloons and piston-cylinder actuators as fluidic actuators. However, bellows or spring-embedded balloons, can be equally good or even better candidates to replace these pistons. In addition, the front balloon 2 inlet can pass inside or through the extensor body 6 as shown in FIG. 13 instead of outside as shown in FIGS. 8 to 11, improving compatibility of the carrying platform 10 by reducing the overall maximum outer diameter.

Another possibility is using a “gauss gun-inspired piston” wherein the piston or another actuator configuration is actuated by electrical current or electromagnetism rather than a hydraulic or pneumatic pressure or pressure that is derived from chemical reaction or reversible chemical or physical reaction. In an embodiment of the carrying platform 10, the piston rod is connected to a strong magnet located inside the cylinder while the cylinder is enwrapped by sections of conductive coil. Running direct current inside these coils will generate a controlled magnetic field inside the cylinder that can be used to induce a very precise actuation. This technology is currently used in anti-aircraft guns for replacing gun power-based bullets allowing a very high rate of fire. However, many other types of more common electromagnetic actuators are available and can be used for this.

In another embodiment of this invention, the carrying platform 10 may comprise magnets and magnet-attracted material, said magnets can be induced to generate motion to carry the catheter 1 or endoscope 1 from outside a patient by control of the movement and location of these magnets by a magnetic field that is induced by a device that is external to the patient. This can be desirable as such configuration does not require channels to connect the platform directly to an external controller. Moreover, this can allow the fabrication of a smaller profile carrying platform 10 at lower costs.

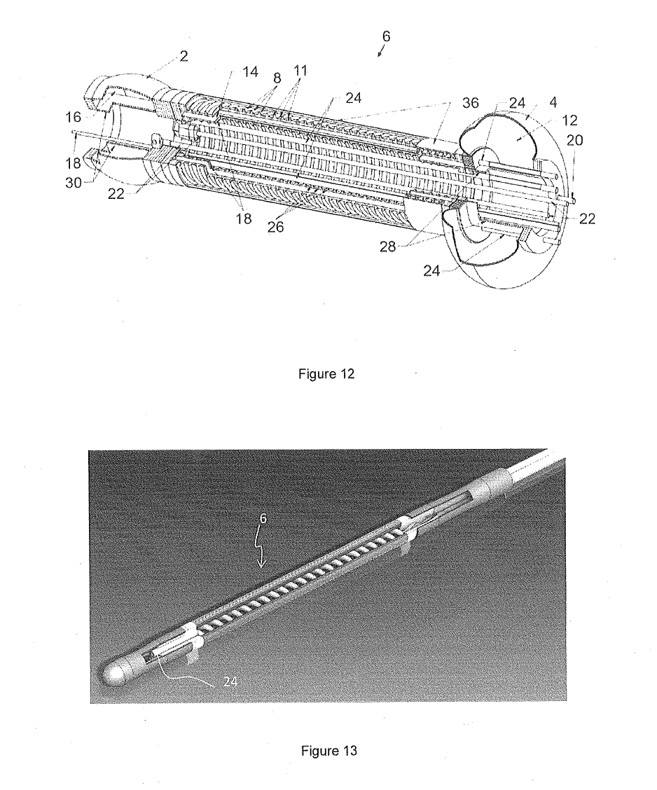

FIG. 12 is a schematic illustration of a hollow fiber-stiffened balloon carrying platform 10 having pressure supply lines that are used as a means to enable three degree of freedom (3-DOF) movement and comprising a front inflatable clamper 2, a rear inflatable clamper 4, and an external elastic protective shell 36 surrounding the middle inflatable extensor or body 6. The body 6 has an inflatable wall comprising an external elastic balloon or layer 10 and an internal elastic balloon or layer defining a space 14 therebetween. Stiffening fibres 8 are embedded into the external elastic layer 11 and a stiffening return spring 26 is embedded, or fibers, can be embedded into the internal elastic layer. The internal elastic layer defines a hollow passage through the body 6. The inflatable cavity 12 between a rigid cylinder and elastic cylinder of the rear clamper 4 is shown in the inflated state with its pressure supply line indicated by the reference numeral 20, while the inflatable cavity 16 between the rigid cylinder and the elastic cylinder of the front clamper 2 is shown in the deflated state. Pressure supply lines 22 of the front clamper 2 are crossing in the hollow section or pass through the hollow body 6 of the carrying platform 10 device and are secured to stiffening rings of both the extensor body 6 and the front clamper 2. Holding in place or pulling one of these lines 22 during inflation of the middle extensor body 6 will cause bending of the carrying platform 10 in the direction of the line held. The motion can be extended into an extension-bending motion if the supply line 22 is slightly released during or after inflation of the extensor body 6. The bending of the device, similarly to a marionette, can be induced by applying pull force on the supply lines 22.

Bending strings or additional supply lines 24 are provided to either act as additional supply lines of the front clamper 2 or the middle extensor body 6 or for an inflatable tip. Additionally they 24 can be used as a payload delivery mechanism. These additional supply lines 24 pass through the hollow section of the carrying platform 10 and are secured to stiffening rings of both the extensor body 6 and the front clamper 2. Holding in place or pulling one of these lines 24 during inflation of the middle extensor body 6 will cause bending of the carrying platform 10 in the direction of the line that is being held. The motion can be extended into an extension-bending motion if the supply line 24 is slightly released during or after the extensor inflation.

The stiffening return spring 26 embedded within the internal elastic balloon of the middle extensor body 6 both increases the pulling force of the carrying platform 10 during contraction, and also prevents collapse of the internal elastic layer of the extensor body 26 during inflation. A rigid cylinder 28 is provided in the interior of the rear clamper 4. This cylinder 28 prevents collapse of the rear clamper 4 into the hollow section of the body 6 during inflation of the rear clamper 4. The cylinder 28 can also include the rear gripper (not shown). A rigid cylinder 30 is provided in the interior of the front clamper 2. This cylinder 30 prevents collapse of the front clamper 2 into the hollow section of the body 6 during inflation of the front clamper. The cylinder 30 can also include the front gripper (not shown). An imaging modality 18, or an additional payload channel is additionally provided that passes through the hollow passage of the navigation carrying platform 10.

The combination of both a spring and a fiber stiffened balloon in the body can be beneficial as it allows a weaker spring with less axial resistance. In addition, having many fibers between the spring wire can improve the effects of axial expansion or extension of the body by blocking any lateral expansion or inflation of the body into gaps formed between the spring after the distance between the spring wires increases as a result of stretching of the spring; the same principle can be achieved by overlapping several layer of fiber stiffened sheet.

The advantage of such a carrying platform 10 as described above with reference to FIG. 12 is that the simplicity of the design results in reduced production costs. Moreover, as no rigid elements are present, the design is safer to use. Most importantly, a hollow design allows insertion of the catheter or endoscope 1 into this gap, thus, the overall diameter of the carrying platform 10 is reduced.

In alternative embodiments, a hollow configuration of the carrying platform 10 may be achieved by linking torus-shaped balloons having supports in their interior. Balloons can be inflated in chain to induce earthworm-like locomotion or be used to make inchworm like locomotion as described thus far. Additional alternative designs may include use of bellows, or a spring-embedded or fiber-embedded balloons in the midsection. Designs that use shape memory, wire-actuated, electroactive actuators, etc. rather than fluidic actuators may also be used. To make a hollow balloon based device, a supporting scaffold can also be used.

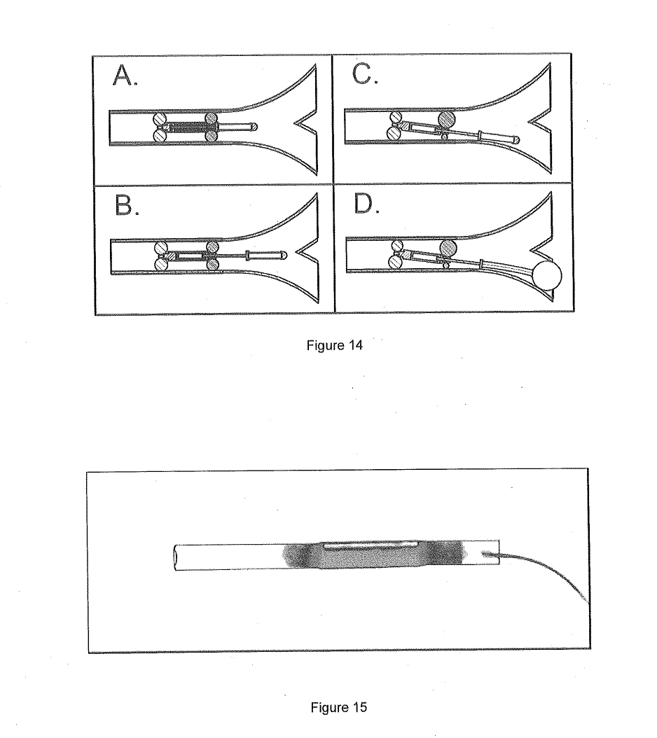

One of the advantages of a hollow carrying platform is applying it for use in procedures that requires continued blood flow or in places like the trachea as it will allow continued breathing. A hollow carrying platform can also be applied as a local pump or blood pump. When there is a problem with blood flow, the balloon in the carrying platform can be activated in sequence and be used to pump blood with contraction movements into an oxygen deprived area or to breakdown clots etc. When attempting to deliver a payload such as a drug, active agent, marker, etc. to a targeted section of the vessel or conduit using a hollow carrying platforms, while this hollow carrying platform is designed to allow fluid flow, it may be beneficial to add a controllable flow blocking mechanism to allow flow blockage, resulting in a better delivery to the required region. Balloons used in the mentioned configurations, and additional balloons specifically added along the supply lines or others, can be used for generation of these sealed-off regions or to manipulate the movement of the carrying platform as shown in FIG. 14. Balloons may also be used to confine the delivered drugs, or agents to a specific location defined by inflation of certain balloons, such as shown in FIG. 15, in which the luminescent material is locally confined between two inflated balloons. In further development of this, the carrying platform 10, or any other device that is able to both deliver a drug and block the biological conduit can be used to seal-off a certain region of the body by utilization of one or more carrying platforms 10 that are used to seal the entrances and exits of a confined system of conduits that supplies this region or organ.

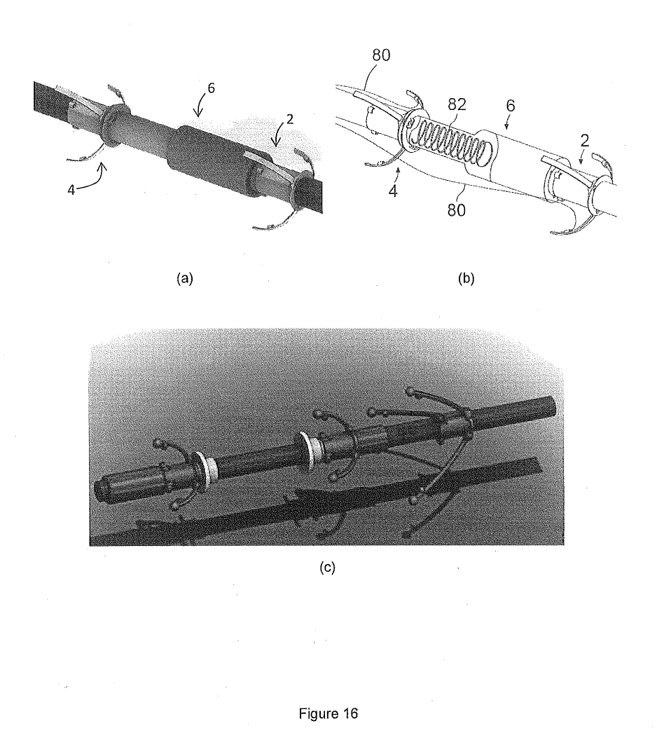

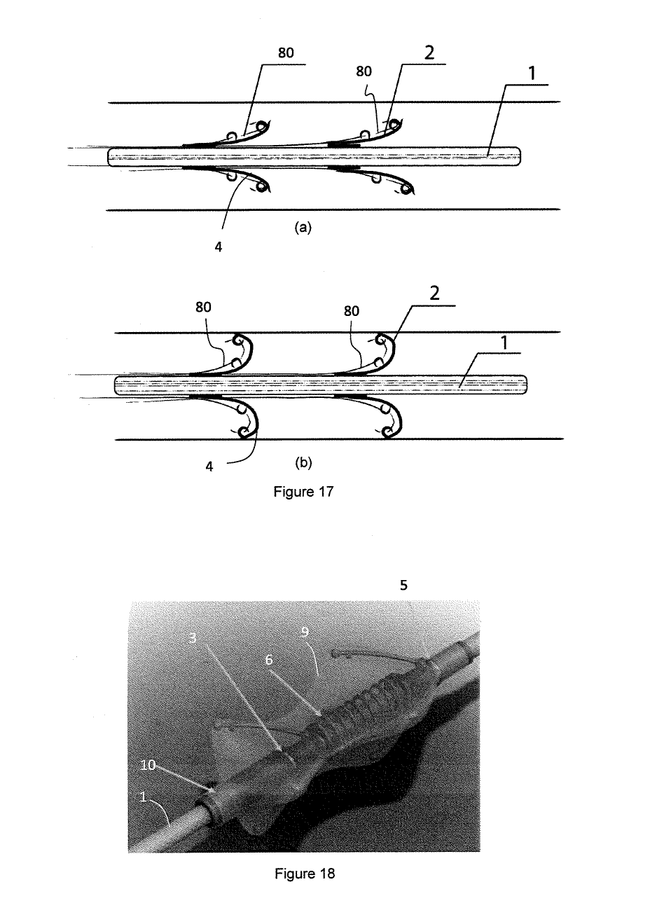

Another implementation of the carrying platform is not to use hydraulic or electrically actuated actuators, but wire-controlled actuators as shown in FIGS. 16 and 17. The wires 80 are used to transfer force to power the actuators, similarly to a marionette. Using wires can dramatically reduce the reaction time of the actuators, device diameter and the manufacturing complexity of the carrying platform 10. Moreover, it is by far the safest possible actuator to use as no leakage risk, heat or high voltage are required for actuation.

As shown in FIG. 16(b), which is a generic example of one possible way to implement a wire actuated device, the clampers 2, 4 comprise arms that can be controlled by pulling the wires 80. When the wire 80 is pulled, the clamper 2 or 4 will be detached from the lumen wall 7. Otherwise, the clamper 2 or 4 will expand when the wire 80 is relaxed so that the arms engage the conduit 7 when the clamper 2 or 4 is at rest. The extensor body 6 is also controlled by balance of forces between a compressible material, repelling/attracting magnets or a spring 82 provided in the body 6 and the wires 80. When the wire 80 is not pulled, the spring 82 will expand the body or keep the main body 6 extended. Pulling another wire (not shown) compresses the spring 82 to contract the main body 6. With this movement repeated, the extensor 6 can realize moving forward and backward via contraction and extension. Besides mechanical pulling, other wires-actuators, such as carbon nanotubes wire actuators and others which are controlled by temperature, charge, current chemical reaction, light, etc. can be used. When actuated, the length of the wires 80 is decreased or increased accordingly to exert motion on a part. Similarly, the wires 80 can be connected to any kind of actuators for controlling their tension.

An alternative embodiment of a wire or cable actuated carrying platform 10 is depicted in FIGS. 17(a) and 17(b) in which the clampers 2, 4 comprise cable actuated linkage mechanisms. At rest, the clampers 2, 4 are disengaged from the conduit 7 such that the carrying platform 10 can be moved by within the conduit 7 by engaging the carrying platform 10 with the device 1 and moving the device 1. When the clampers 2, 4 are actuated by the cable 80, the clampers 2, 4 open up to engage the conduit 7, thereby securing the carrying platform 10 to the conduit 7.

FIG. 18 shows a perspective view of an exemplary wire actuated carrying platform 10 covered with an elastic layer 9, and in which the device engaging mechanism or front and rear grippers, 53 comprise elastic rings 3, 5 configured to grip the device or catheter 1. The elastic rings 3, 5 are looped around by a string that in tension squeezes the elastic ring 3, 5 against the catheter 1 to grip the catheter 1.

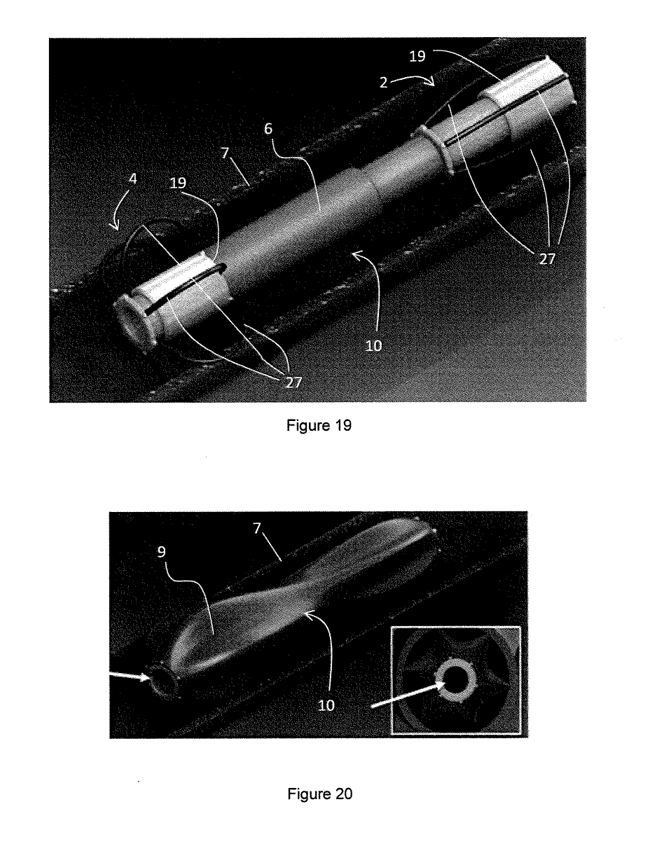

A further exemplary embodiment of the carrying platform 10 is shown in FIG. 19 in which the front and rear clampers 2, 4 each comprise a plurality of bendable rods 27 disposed around the body 6. For each clamper 2 or 4, a first end of the rods is attached to the body while a second end of the rods is attached to a sleeve 19 configured to slidably engage the body 6. Bending of the rods 27 causes a central portion of the rods 27 to engage the conduit 7, and may be actuated by wires or be EAP, DEA or SMA actuated. Each of the rods 27 may be made of a bimetal, for example. FIG. 20 shows the carrying platform 10 of FIG. 19 covered with a protective elastic layer 9. Notably, the carrying platform 10 when deployed in a conduit 7 such as a blood vessel does not fully block the blood vessel so as to allow for continued blood flow through the blood vessel 7. The arrow indicates the longitudinal through hole of the carrying platform 10 itself through which a device 1 such as a catheter 1 may be passed through to be gripped by the carrying platform 10.

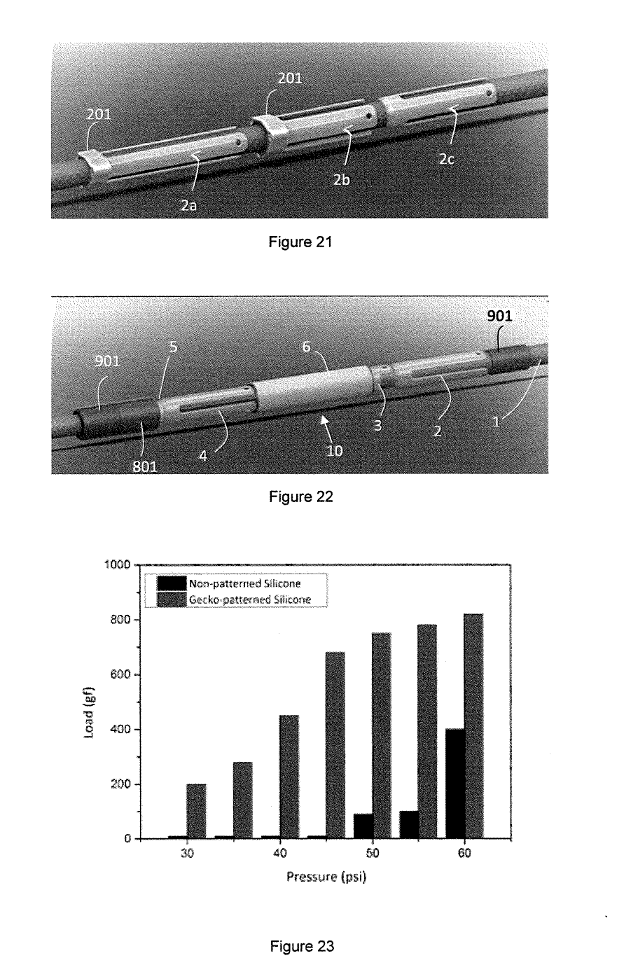

FIG. 21 shows three variations 2a, 2b, 2c of a wire actuated clamper 2 or 4 of the carrying platform 10. The first variation 2a is a long clamper 2a that can fit very large diameters of up to 30 mm The second variation 2b is a shorter clamper that can fit diameters of up to 20 mm. Both the first and second variations 2a, 2b comprise alignment rings 201. The third variation 2c is a clamper that does not contain any alignment ring, thereby allowing easy manufacturing by either fento-lasering or EDM of a metal tube or machining a metal sheet and then rolling it to form the final tubular clamper body or by popup manufacturing techniques and the like. The aligners for the third variation can come later or not at all if the pressure points can be absorbed by the alignment point on the part before. These variations 2a, 2b, 2c of clampers 2, 41ie flat in their native state, and only open up to engage the conduit 7 when the wire is pulled, so that if something goes wrong, the carrying platform 10 will not become lodged inside a blood vessel, for example.

FIG. 22 shows a further exemplary carrying platform 10 of the present invention engaging a device 1 such as a catheter 1. The device engaging mechanism comprises a rear gripper 5 having an elastic ring with wire looped around it as described above with reference to FIG. 18, such that when the wire is pulled, it presses onto the ring which presses against the catheter 1, thereby holding the structure 10 in place relative to the catheter 1. The device engaging mechanism also comprises a front gripper 3 having a structure very similar to the clamper 2, 4 that has a configuration as described with reference to FIG. 21. In this front gripper 3, there is a little bulge (not shown) that engages the catheter 1 at rest. When the wire is pulled, the bulge is released from the catheter 1 and the gripper 3 is turned off to disengage from the catheter 1. In this design, the gripper 3 is natively turned on which eliminates the risk of the carrying platform 10 being released and removed from the catheter 1 should anything go wrong.

The exemplary carrying platform 10 shown in FIG. 22 also comprises a wire gathering portion 801 that is configured to take in all the wires (not shown) that actuate the clampers 2, 4 and grippers 3, 5 from a single point and distribute them via channels that are inside itself. In this portion 801 also must be installed a mechanism (not shown) that deals with the fact that in most catheters, the diameter of the tip is smaller than the diameter of the back. This mechanism can comprise, among many other options, hairs, like a brush, that are covered with omniphobic coating in order to prevent blood from passing into the cavity of the wire gathering portion 801. The extensor or body 6 is not a spring or pairs of repelling magnets but a compressible material made by very elastic dragonskin silicon that is infused with air bubbles. Securing portions 901 provided at a front and rear end of the carrying platform are configured to be fused to an elastic gecko covering sheet or layer 9, the purpose of which has been described above.

To improve navigation and specifically to bolster the ability of the carrying platform to perform turns (in a junction, etc.) or pass clogged surfaces, a magnet or a ferromagnetic material or any type of material that can be effected by magnetic field can be provided, such as installed at its tip, or at any other suitable location on the carrying platform. This will allow application of an external magnetic field that can enable faster movement and sharper turns and higher force while advancing the carrying platform that is guiding the catheter 1 or endoscope 1.

In addition to improving navigation capability, magnets or compressible materials can also be used instead of a return spring in the extensor body to contract the body, allowing easier miniaturization of the piston cylinder if that is being used as an actuator. The same magnets can be used as a navigation aid as described above.

To achieve bending motion with balloons, in addition to the above described methods, overlap while rolling or wrapping an elastic sheet over a spring or overlapping the rolled elastic sheet with fibers embedded in it or using a material with stiffness or elasticity that can be controlled can be used to induce a bending motion in the body. If the overlap is not symmetric, having a part of the roll slightly thicker, the balloon of the body will bend in this thicker direction while expanding. The same principle can be achieved if a fiber or a flexible rod is added to one side of the balloon of the body in the direction of expansion. It is noteworthy to mention that while thicker sections will allow significant bending of over 360 degrees, if a spring is used, these will be harder to achieve as the spring stabilizes the structure, which in turn can be a benefit.

In addition to the above stiffening-induced bending, directional bending can be achieved by including structures with electroactive material or other smart materials with the carrying platform. Such materials can actively induce bending but can also operate passively by controllably adjusting their stiffness.

Another possible configuration to allow bending of the carrying platform is having a rotatable bending tip, eliminating the need of linking several tips. Having several separate compartments within the same tip can also enable multidirectional bending capacity, while having such said compartment on the sides of the extensor body can also induce bending without the need of an additional tip.

Providing an additional clamper at the end of the bending tip, or providing the additional clamper after the bending apex can result in a better design by allowing the carrying platform to clamp and immobilize at the desired location, thus stabilizing the operation.

Where the body is actuated using a piston mechanism or other actuators, the rear clamper does not necessarily need to be mounted behind the piston body. Having the rear clamper mounted in front on the cylinder near the piston seal will result in shorter distance between the rear and the front clamper which will enable higher degree of motion in bending and may reduce the cross-sectional diameter of the carrying platform. Controlled rotation of the piston rod (by pumping back and forth, for example) will allow the usage of a single bending tip for a full 3-DOF. If the rear clamper is located on the flexible pressure supply tubes in addition to rather than on the rigid piston—a higher degree of bending will allow the carrying platform to bend under pressure.

Despite the diversity of configuration described herein that suggest possible ways to enable controlled pitch and turn of the carrying platform, one must understand that the big advantage of this invention is that it can be added onto conventional catheters. If the carrying platform is used for catheterization without a catheter, meaning it is being operated as a catheter replacement, these bending abilities will become crucial. The main advantage and novelty of this invention is that it allows an improvement of conventional catheters. The bending and rotation of catheters is currently well established and in many cases performing very well. As such, the carrying platform does not need to be equipped with these abilities but only abilities that help in the forward and backward propulsion of the catheter 1 that it carries. The bending and rotation will come from the catheter 1 itself.

Adding gecko, treefrog, mushroom, beetle, or any kind of structural based or chemical-based or physics-based adhesive or any combination of adhesives to the front and rear clampers of the carrying platform is of a particular advantage. This family of adhesives is well studied and easy to fabricate (hemocompatible elastic gecko-like and beetle-like sheets made by medical grade silicone are commercially available at low prices). The gecko adhesive works best in a way that it adheres best when a lateral force is applied. It is a friction-based adhesive. This means that when the front or rear clamper presses against the wall of the conduit and the extensor body applies a lateral force as it extends or contract during the motion cycle, the gecko inspired adhesive will provide extra grip to the engagement of the front or rear clamper with the conduit and compensate friction deficit in slippery, or smooth environments. FIG. 23 shows a graph of load vs pressure for gecko-patterned and non-patterned silicone. The gecko-patterned silicon clearly shows significantly greater pressure given the same load. This allows the application of less force against the sensitive endothelium in order to achieve adequate grip. While the adhesion benefits are activated by the lateral force during axial motion of the carrying platform, there are no adhesion effects when the front or rear clamper releases from the wall as no lateral force is applied on the adhesive during this motion. This is of great benefit as the adhesive will not hinder the performance of the actuator. Moreover, in delicate environments such as a blood vessels, if the front and rear clampers need to be forcefully pulled from the wall, the gecko adhesive does not induce any adhesion effect while being detached and therefore risk of damage during detachment of the front or rear clampers is eliminated.

Preferably, the carrying platform may be wirelessly powered using such wireless powering sources such as RF charging, magnetic induction, focused ultrasound, battery, utilizing the energy derived from the flow surrounding the carrying platform, and so on.

In addition to using the carrying platform's own fluidic system, therapeutic or active payloads carried by the carrying platforms described above can be numerous: photocatalytic and electrocatalytic arms and stents can be used, possibly operated by the carrying platform's own imaging modality by switching to a different wavelength; active-nanoparticles and medicine can be entrapped using an encapsulating mesh coating the internal part of the hollow carrying platform or located on a separate appendix or on the covering sheet of the carrying platform or the external areas of the carrying platform. These particles can be released possibly by changing the wavelength of the imaging modality. For example, pulling an imaging fiber to different locations relative to the carrying platform can enable more release flexibility, several agents, etc. Alternatively, a releasing/activating agent can be introduced via the carrying platform's fluidic system to initialize the reaction. Another possibility is to utilize focused ultrasound to release the trapped particles. Addition of separate compartments containing drugs or agents onto the carrying platform is also a possibility, these compartments will release the drug or agent particles when required.

The carrying platforms described above can be equipped with pressure sensors, such as on the clampers or extensor body, etc. Thin and flexible sensors with micron sensing resolution can be installed on the outer section of the clamper, directly contacting the walls or conduit. While this can be used as a safety measure to prevent over pressing sensitive walls, the application can also be extended as a sensory module, much like an artificial finger. This can enable detection of arterial stiffness, mapping of arterial plaques, detecting calcification of tubes etc. and even be used as an early warning for future arterial disease. If a wire actuated system is used, the wire can serve as an instant force sensor. The force exerted in order to pull the wires can be measured and directly translated to the force that is needed in order to activate the clamper, this can be instantly shown when contact with the artery is made.

In addition to moving catheters, the suggested carrying platform can be used as search and rescue robots, navigating between pieces of rubble; as inspection and maintenance carrying platforms for piping systems and the like, in such cases the flow from the pipe can be used to power the carrying platform and make it completely wireless; as swimming carrying platforms with particular advantage in high viscosity liquids; as digging robots; as carrying platforms able to navigate between walls, such as between two buildings. This might be of use in defense applications, where reconnaissance is needed in urban warfare, or for the deployment of weaponry or as surveillance/reconnaissance carrying platforms via piping systems or at the higher floors of a building in a very densely constructed area, where images from conventional drones are harder to obtain; the device can also be used in aircon conduits or any other conduits where access is needed. In addition, and especially if an adhesive such as gecko, electro-activated or other types of adhesives are added, the carrying platforms are not limited to navigation between walls. The carrying platforms are capable of moving on a single horizontal or tilted wall and surfaces due to friction differences between retracted and contracted clampers.

All the carrying platforms described above can be extended to applications where they navigate to and stay in a targeted location. Upon deployment they can be used for slow release of therapeutic or functional agents, as stents, lab-on-chip sensors etc. Using a clearance seal for the piston actuator enables a steady and controlled flow of saline-anticoagulant mix into a blood vessel to prevent clotting formation when using the carrying platform in a blood vessel. The targeted delivery of drugs, using leaks in the carrying platform as described above can be further expanded into other applications, such as pipes or any conduit system maintenance. Active agents can be locally or continuously delivered using the described carrying platform. In the human body, the carrying platform can be released from the catheter it is carrying and be deployed locally to gather information and or deliver payload in a controlled manner. The carrying platform is hollow and allows the continuation of the biological flow. The carrying platform can be removed using a catheter or other surgical operation or be absorbed by the body if it is made from biodegradable materials. One can also envision that the carrying platform may be incorporated with cell differentiation factors to serve as a scaffold for organ regeneration such as blood vessel, heart tissue, bone, muscle, etc.

Whilst there has been described in the foregoing description exemplary embodiments of the present invention, it will be understood by those skilled in the technology concerned that many variations in details of design, construction and/or operation may be made without departing from the present invention. For example, while the clampers have been described as each possibly comprising a balloon, other known mechanisms may be provided as the clampers so long as actuating of the clamper engages a portion of the carrying platform with the conduit, for example, fluidic actuators or actuators that are controlled by physical and chemical reactions, such as actuators that are controlled by melting and solidifying wax, magnets from outside the body, shape memory materials, electromagnetic, electroactive, ionic polymers, bimorph or bimetallic strips, dielectric elastomers, or even biological muscles controlled by electric stimuli. While the grippers have been described as each possibly comprising a sleeve or a balloon, other known mechanisms can be used for the grippers, so long as actuating of the gripper causes the gripper to grip the device, for example, electromagnetic, wires, bimetallic or SMA strips, or tissue engineered muscles that will be extremely biocompatible for long uses as it can be powered by nutrients from the blood.

1. A carrying platform for moving a device within a conduit, the carrying platform comprising:
a body configured to be moveable within the conduit;
a number of clampers provided on the body and configured to releasably engage the conduit for immobilizing the carrying platform relative to the conduit; and
a device engagement mechanism provided on the body and configured to releasably engage the device.

2. The carrying platform of claim 1, wherein the device engagement mechanism comprises at least a first set of wheels configured to effect relative movement between the carrying platform and the device and to allow the carrying platform to move along a length of the device within the conduit.

3. The carrying platform of claim 1, wherein
the body comprises an actuating mechanism configured to selectably extend and contract the body;
the number of clampers comprise a front clamper and a rear clamper provided spaced apart on the body, the front clamper and the rear clamper each configured to independently engage the conduit such that a front portion of the body is immobilised relative to the conduit when the front clamper engages the conduit and a rear portion of the body is immobilised relative to the conduit when the rear clamper engages the conduit; and
the device engagement mechanism comprises a front gripper and a rear gripper provided spaced apart on the body, the front gripper and the rear gripper each configured to independently grip the device.

4. The carrying platform of claim 3, wherein the carrying platform is configured such that engaging the rear clamper with the conduit and extending the body when the front clamper is disengaged with the conduit results in forward movement of the front portion in the conduit, wherein engaging the rear clamper with the conduit and contracting the body when the front clamper is disengaged with the conduit results in backward movement of the front portion in the conduit, wherein engaging the front clamper with the conduit and extending the body when the rear clamper is disengaged with the conduit results in backward movement of the rear portion in the conduit, and wherein engaging the front clamper with the conduit and contracting the body when the rear clamper is disengaged with the conduit results in forward movement of the rear portion in the conduit.

5. The carrying platform of claim 4, wherein when the rear gripper is not gripping the device, movement of the front gripper in the conduit when gripping the device correspondingly moves the elongate device in the conduit; and wherein when the front gripper is not gripping the device, movement of the rear gripper in the conduit when gripping the device correspondingly moves the device in the conduit.

6. The carrying platform of claim 3, wherein the front gripper and the rear gripper each comprise one of: a sleeve made of a shape memory material, a sleeve actuated by wire to grip the elongate device, and a balloon configured to grip the device when inflated.

7. The carrying platform of claim 1, wherein the body has an inflatable wall comprising an external elastic layer and an internal elastic layer defining a space therebetween, wherein the external elastic layer is stiffed with fibres and the internal elastic layer is stiffened by a spring, and wherein the internal elastic layer defines a hollow passage through the body.

8. The carrying platform of claim 7, wherein the front clamper is provided with pressure supply lines that pass through the hollow passage, the pressure supply lines configured to cause bending of the body to occur when the inflatable wall is inflated and one of the pressure supply lines is pulled or held in place.

9. The carrying platform of claim 7, further comprising an imaging modality that passes through the hollow passage.

10. The carrying platform of claim 1, wherein the body is hollow and comprises a bellow.

11. The carrying platform of claim 3, wherein the body comprises a cylinder actuator having a piston-in-cylinder configuration, wherein the front clamper is provided on the piston and the rear clamper is provided on the cylinder.

12. The carrying platform of claim 1, wherein the front clamper and the rear clamper each comprise a balloon configured to engage the conduit when inflated.

13. The carrying platform of claim 1, wherein the front clamper and the rear clamper each comprise arms that engage the conduit when at rest, the arms being actuated by a wire to disengage with the conduit when the wire is pulled.

14. The carrying platform of claim 1, wherein the front clamper and the rear clamper each comprise arms that are disengaged with the conduit when at rest, the arms being actuated by a wire to engage with the conduit when the wire is pulled.

15. The carrying platform of claim 13, further comprising a spring provided in the body wherein pulling a further wire compresses the spring to contract the body.

16. The carrying platform of claim 1, wherein gecko adhesive is provided on the number of clampers at where the number of clampers are configured to engage the conduit.

17. The carrying platform of claim 1, wherein the carrying platform is configured to be connected to a further carrying platform of any preceding claim.

18. The carrying platform of claim 1, wherein the carrying platform is covered with an elastic layer configured to protect the carrying platform from substances found within the conduit.
